(19) United States
(12) Patent Application Publication
(10) Pub. No.: US 2022/0068460 A1
(43) Pub. Date: Mar. 3, 2022

(54) METHOD FOR EVALUATING NUTRIENT CONTENT ON A CALORIE-SCALED BASIS

(71) Applicant: WISEcode, LLC, Reno, NV (US)

(72) Inventors: Peter M. Castleman, Reno, NV (US); Shavawn M. Forester, Zephyr Cove, NV (US)

(21) Appl. No.: 17/464,785

(22) Filed: Sep. 2, 2021

Related U.S. Application Data
(60) Provisional application No. 63/073,595, filed on Sep. 2, 2020.

(51) Int. Cl. G16H 20/60; G16H 50/70; G16H 10/60

(52) U.S. Cl. CPC G16H 20/60; G16H 10/60; G16H 50/70

(57) ABSTRACT

Methods and an associated system for evaluating nutrient content of a food item are provided. In some embodiments, the method includes receiving a nutrient data set for the food item, the nutrient data set including a plurality of nutrients in the food item and an amount of each nutrient per serving of the food item. The method includes generating a scaled nutrition score based on the nutrient data set, the scaled nutrition score being scaled to a calorie reference value. The method further includes generating a nutrition quality ratio by dividing the scaled nutrition score by a number of calories per serving of the food item.

### CROSS-REFERENCE TO RELATED APPLICATION

This application claims the benefit of U.S. Provisional Patent Application No. 63/073,595, filed on Sep. 2, 2020, entitled “METHOD FOR EVALUATING NUTRIENT CONTENT ON A CALORIE-SCALED BASIS,” which is incorporated herein by reference in its entirety.

### BACKGROUND

Dietary choices have a significant impact on health and wellbeing. However, it may be difficult for individual consumers to determine which food items are likely to be beneficial to their health. Although federally-mandated food labels provide some information on the nutritional content of food products, the information on these labels is limited to a small set of nutrients and omits many other nutrients that are essential for life and health. Additionally, it may be difficult for laypersons to assess how the nutritional information presented on a food label correlates to the overall nutritional quality of the food item.

### BRIEF DESCRIPTION OF THE DRAWINGS

FIG. 1 is a network diagram illustrating an exemplary computing environment in which a nutrition evaluation system operates.

FIG. 2 is a flow diagram illustrating an exemplary process for evaluating nutrient content of a food item.

FIG. 3 is a flow diagram illustrating an exemplary process for generating a scaled nutrient score.

The techniques introduced in this disclosure can be better understood by referring to the following Detailed Description in conjunction with the accompanying drawings.

### DETAILED DESCRIPTION

Methods for evaluating the nutrient content of food items and providing quantitative nutrition scores that are scaled to calorie intake are disclosed herein. The method includes receiving a nutrient data set that characterizes a food item. The nutrient data set includes a plurality of nutrients in the food item (e.g., vitamins, minerals, amino acids, fatty acids, fiber, sugar, phytonutrients) and an amount of each nutrient per serving of the food item. The method also includes generating a nutrition score based on the nutrient data set and scaling the nutrition score to a calorie reference value (e.g., a value representing an average and/or recommended daily calorie intake, such as 2000 calories). In some embodiments, the scaled nutrition score is generated based on point values computed from different nutrients or different subsets of the nutrients. For example, the scaled nutrition score may be generated based on one or more of the following: comparisons between the amounts of certain nutrients per serving of the food item and the recommended intake values for those nutrients; ratios between the amounts of certain nutrients; and/or comparisons between the amounts of certain nutrients and one or more threshold values. The method further includes dividing the scaled nutrition score by a number of calories per serving of the food item to determine a nutritional quality ratio for the food item. The nutritional quality ratio provides a standardized, quantitative rating that can be used to represent the overall nutrient content per calorie of a food item.

The methods described herein condense complex nutritional data into a simple, universal metric, thus allowing individual consumers to make informed dietary choices to improve their health and wellness. Additionally, the disclosed methods can be applied to evaluate the nutrient content of hundreds or thousands of food items with highly diverse compositions. Because of the complexity involved in compiling and processing large amounts of food and nutritional data to analyze many different types of food items—the nutrition evaluation system disclosed herein maintains nutrient data for more than 35,000 ingredients—the techniques described herein are not suitable for implementation using manual approaches.

Various embodiments of the present technology will now be described. The following description provides specific details for a thorough understanding and an enabling description of these embodiments. One skilled in the art will understand, however, that the present technology may be practiced without many of these details or with alternative approaches. Additionally, some well-known structures or functions may not be shown or described in detail so as to avoid unnecessarily obscuring the relevant description of the various embodiments. The terminology used in the description presented below is intended to be interpreted in its broadest reasonable manner, even though it is being used in conjunction with a detailed description of certain specific embodiments of the present technology.

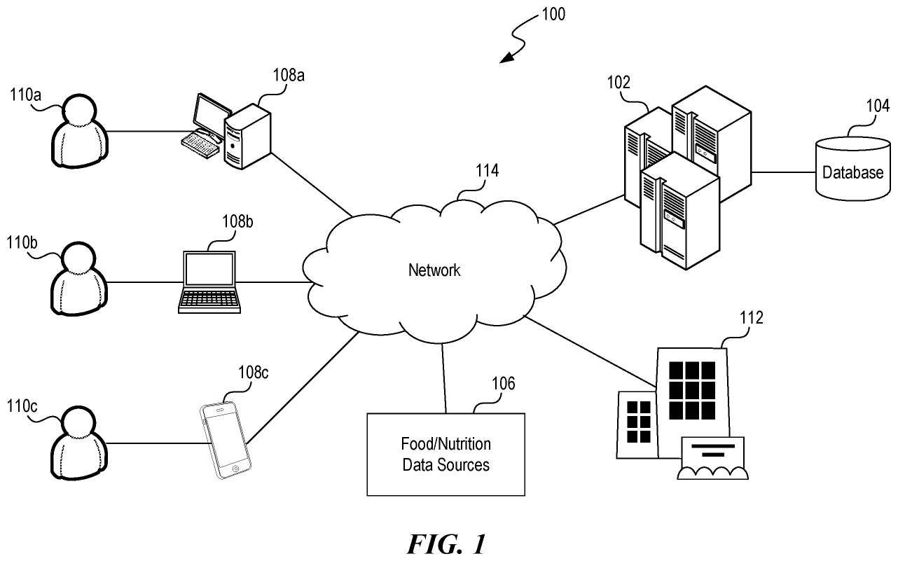

FIG. 1 is a network diagram illustrating an exemplary computing environment 100 in which a nutrition evaluation system operates. Although not required, aspects of the system are described in the general context of computer-executable instructions, such as routines executed by a general-purpose computer, a personal computer, a server, or other computing system. The system can also be embodied in a special purpose computer or data processor that is specifically programmed, configured, or constructed to perform one or more of the computer-executable instructions explained in detail herein. Indeed, the term “computer” and “computing device,” as used generally herein, refer to devices that have a processor and non-transitory memory, like any of the above devices, as well as any data processor or any device capable of communicating with a network. Data processors include programmable general-purpose or special-purpose microprocessors, programmable controllers, application-specific integrated circuits (ASICs), programming logic devices (PLDs), or the like, or a combination of such devices. Computer-executable instructions may be stored in memory, such as random access memory (RAM), read-only memory (ROM), flash memory, or the like, or a combination of such components. Computer-executable instructions may also be stored in one or more storage devices, such as magnetic or optical-based disks, flash memory devices, or any other type of non-volatile storage medium or non-transitory medium for data. Computer-executable instructions may include one or more program modules, which include routines, programs, objects, components, data structures, and so on that perform particular tasks or implement particular abstract data types.

Aspects of the system can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or subroutines may be located in both local and remote memory storage devices. Aspects of the system may be distributed electronically over the Internet or over other networks (including wireless networks). Those skilled in the relevant art will recognize that portions of the system may reside on a server computer, while corresponding portions reside on a client computer.

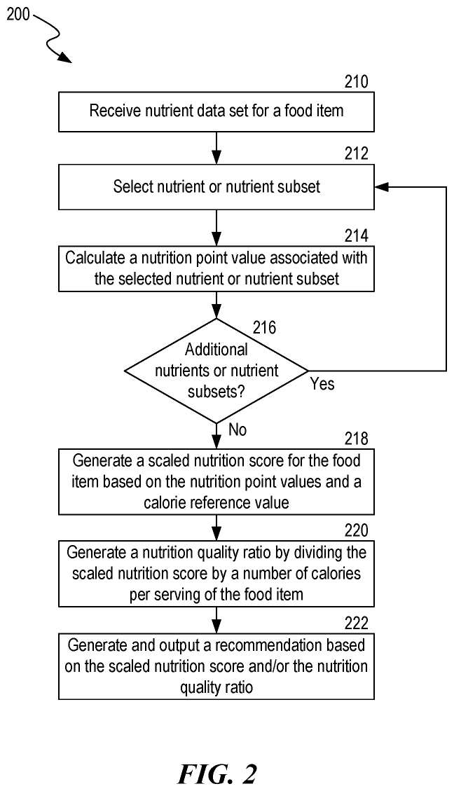

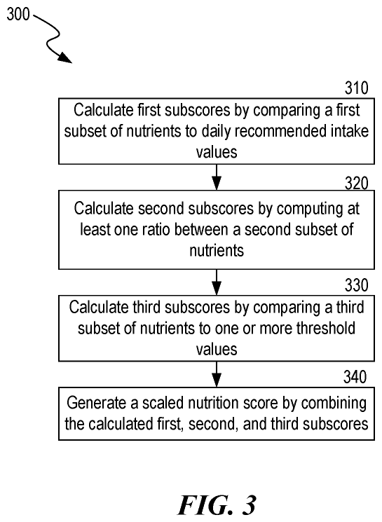

The environment 100 includes one or more server computers 102 (“servers”) that are operably coupled to a database 104, one or more third party food and/or nutrition data sources 106 (“food/nutrition data sources”), a plurality of user devices 108a-c that are respectively associated with a plurality of users 110a-c, and one or more food manufacturers or distributors 112. Aspects of the nutrition evaluation system may be implemented in and/or practiced by the servers 102 and/or user devices 108a-c. In some embodiments, for example, the servers 102 are configured to evaluate the nutrient content of one or more food items. The evaluation is performed based on data (e.g., user data, food/nutrition data) received from the database 104, food/nutrition data sources 106, and/or the user devices 108a-c. The evaluation results (e.g., scaled nutrition scores, nutrition quality ratios, recommendations) can be stored in the database 104 and made available to multiple different parties for a wide range of uses. For example, the evaluation results can be transmitted to the user devices 108a-c and displayed to the users 110a-c, e.g., to assist the users 110a-c in making healthy dietary choices, meal planning, or other health and nutrition-related activities. Users may include consumers, food preparation professionals, medical professionals, researchers and other academics, or any other party that would benefit from nutrition data that facilitates the assessment of foods and food choices. The evaluation results can also be provided to food manufacturers or distributors 112 for purposes of product labeling and advertising. An additional description of the processes for evaluating nutrient content is provided below with respect to FIGS. 2 and 3.

In the illustrated embodiment, the servers 102 communicate directly with the database 104 (e.g., via wired or wireless communication techniques), and communicate with the food/nutrition data sources 106 and user devices 108a-c via a network 114. The network 114 can be a LAN or a WAN, and can include wired or wireless network elements. For example, the network 114 can be the Internet or some other public or private network. In other embodiments, the computing environment 100 can be configured differently, e.g., the servers 102 can communicate directly with the food/nutrition data sources 106 and/or the user devices 108a-c, the database 104 can be connected to the servers 102 via the network 114, etc. The term “database” should be broadly interpreted to include both relational databases as well as non-relational databases such as a flat-file. Databases may be locally maintained or remotely accessed via, for example, a cloud data storage service provider.

The food/nutrition data sources 106 can be any third-party data source that provides information relevant to food and/or nutrition. For example, the food/nutrition data sources 106 can be operated by or otherwise associated with a government agency (e.g., the U.S. Food and Drug Administration (FDA), the U.S. Department of Agriculture (USDA)), a food manufacturer, a research institution, or any other suitable entity. The food/nutrition data sources 106 can provide food/nutrition data including any of the following: food composition data (e.g., lists of ingredients, known or estimated amounts of ingredients, lists of nutrients in food items, known or estimated amounts of nutrients in food items, number of calories per serving), nutrient content data for ingredients (e.g., lists of nutrients in ingredients, known or estimated amounts of nutrients in ingredients), food label data, dietary guidelines such as recommended intake values (e.g., recommended minimum, maximum, or average daily intake values; dietary reference intake (DRI) values; recommended daily allowance (RDA) values; adequate intake (AI) values), estimated energy requirements (e.g., number of calories per day based on age, gender, height, weight, physical activity level, pregnancy and lactation status, etc.), food safety data, research data, clinical studies, and the like.

The servers 102 can query the food/nutrition data sources 106 to retrieve food/nutrition data for use in the various processes described herein (e.g., via API calls or other automated, semi-automated, or manual data retrieval operations). The food/nutrition data can be stored in the database 104. For example, the database 104 can store data for hundreds or thousands of different foods and/or data for tens or hundreds of different nutrients. In some embodiments, the database 104 maintains a compilation of nutrient content data for at least 10,000, 20,000, 30,000, 40,000, or 50,000 different ingredients. Without such a robust database of ingredient nutrient data, the nutrition evaluation system would be unable to generate nutrient scores associated with the constantly expanding number of food products on the market. Optionally, different food/nutrition data sources may provide data in different formats (e.g., based on the particular software and/or hardware platform used), and the servers 102 can convert the data into a standardized format for storage in the database 104. The servers 102 can periodically update the food/nutrition data stored in the database 104 (e.g., at predetermined time intervals, when updates are pushed by the food/nutrition data sources 106, when the servers 102 receive requests from the user devices 108a-c, etc.).

The users 110a-c interface with the servers 102 via their respective user devices 108a-c. The users 110a-c can include individual consumers as well as organizations that use food/nutrition data in their operations (e.g., food manufacturers, food service providers, healthcare providers, etc.). The user devices 108a-c can include any suitable computing device, such as mobile devices (e.g., smartphones, tablets), wearable devices (e.g., smartwatches, fitness monitors), laptop computers, desktop computers, and the like. Although FIG. 1 illustrates three user devices 108a-c, the computing environment 100 can include any number of user devices (e.g., hundreds, thousands, tens of thousands, or hundreds of thousands of user devices).

In some embodiments, the user devices 108a-c send user requests to the servers 102, such as requests for food/nutrition data, nutrition evaluation results, recommendations, etc. For example, a user may initiate a request by inputting a name of a food item (e.g., “kale,” “vanilla ice cream,” “red wine”), selecting the food item from a list or drop-down menu, taking an image of the food item, taking an image of a food label associated with the food item, etc. Optionally, users can utilize the user devices 108a-c to transmit other data to the servers 102 that may be used in the nutrition evaluation techniques described herein, such as demographic data (e.g., age, gender, race, ethnicity), medical data (e.g., height, weight, body mass index (BMI), physical activity level, food intolerances or allergies, pregnancy and lactation status, medications, disease or conditions, medical history, familial medical history, genetic risk factors), dietary data (e.g., data of previous, current, or planned future meals; dietary preferences or restrictions), health goals (e.g., a target weight), or any other relevant input data. Requests and/or other data may be received from the user devices 108a-c via a user interface, API call, or other data communication technique. The servers 102 can store the data received from the user devices 108a-c in the database 104. Optionally, in embodiments where the different user devices 108a-c provide data in non-standardized formats (e.g., depending on the software and/or hardware platform of the user device), the servers 102 can convert the information to a standardized format suitable for use in the various operations described herein (e.g., evaluating the nutrition content of a food item, generating recommendations relating to food and nutrition).

The servers 102 can respond to the user requests by generating and sending nutrition evaluation results to the user devices 108a-c, as discussed in detail below with respect to FIGS. 2 and 3. The nutrition evaluation result can include a quantitative score or rating for a food item (e.g., a scaled nutrition score, a nutrition quality ratio), a qualitative score or rating for a food item (e.g., healthy or unhealthy; “green,” “yellow,” “red,” “black”), recommendations regarding the food item (e.g., “eat,” “eat with caution,” “do not eat”), recommendations regarding the user's overall diet or health (e.g., “eat more green leafy vegetables,” “eat less sugar”), notifications (e.g., “you've consumed X calories today”), alerts, reminders, or any other relevant output data. In some embodiments, the servers 102 dynamically generate the nutrition evaluation result when user requests are received. In other embodiments, the servers 102 can preemptively generate and store nutrition evaluation results for different food items in the database 104, independently of user requests. For example, the database 104 can store nutrition evaluation results for hundreds or thousands of different food items. When a user request is received, the servers 102 can retrieve the relevant results from the database 104 and send the results to the appropriate user device. The servers 102 can periodically update the results stored in the database 104, e.g., at predetermined time intervals, when updated food/nutrition data is available, if there are changes in the evaluation algorithms used to calculate the results, etc.

FIG. 2 is a flow diagram illustrating an exemplary process 200 that is executed by the nutrition evaluation system for evaluating the nutrient content of a food item. The process 200 can be performed by any embodiment of the nutrition evaluation system and associated devices described herein. For example, the process 200 can be performed entirely by the servers 102 of FIG. 1, entirely by one or more of the user devices 108a-c of FIG. 1, or by a combination of the servers 102 and user devices 108a-c.

The process 200 begins at block 210 with receiving a nutrient data set for a food item (also known as a “nutrient profile” of the food item). The nutrient data set includes a listing of a plurality of nutrients in the food item and an amount of each nutrient in the food item (e.g., an amount per serving of the food item or a total amount). In some embodiments, the nutrient data set includes data for one or more categories of nutrients, such as vitamins, minerals, proteins, amino acids, fats, fatty acids, carbohydrates, fiber, phytonutrients, and/or water. For example, the nutrient data set can include any of the following nutrients: vitamin A, vitamin B1, vitamin B2, vitamin B3, vitamin B5, vitamin B6, vitamin B7, vitamin B9, vitamin B9, vitamin B12, vitamin C, vitamin D, vitamin E, vitamin K, choline, calcium, chromium, copper, iodine, iron, magnesium, manganese, molybdenum, phosphorus, potassium, selenium, zinc, chloride, sodium, histidine, isoleucine, leucine, lysine, methionine, cysteine, phenylalanine, tyrosine, threonine, tryptophan, valine, unsaturated fat, trans fat, saturated fat, cholesterol, omega-3 fatty acid, alpha-linolenic acid, omega-6 fatty acid, linoleic acid, fiber, sugar, starch, flavonoids, carotenoids, or polyphenols.

In some embodiments, the nutrient data set is an existing data set that is retrieved from a suitable data source or sources (e.g., database 104 and/or food/nutrition data sources 106 of FIG. 1). Alternatively, the nutrient data set can be computed from an ingredient list for the food item. The ingredient list can be determined from a food label and/or from food composition data for the food item (e.g., based on food data from a suitable data source). In such embodiments, the process 200 can further include determining the nutrient content of each ingredient in the ingredient list (e.g., based on nutrient data from a suitable data source), then summing the nutrient content across all of the ingredients to determine the amounts of each nutrient in the food item. For example, the nutrient content of a food item may be determined from a food label in accordance with the techniques described in Kim, J. and Boutin, M., “Estimating the Nutrient Content of Commercial Foods from their Label Using Numerical Optimization,” in New Trends in Image Analysis and Processing-ICIAP 2015 Workshops 309-316 (Murino V., Puppo E., Sona D., Cristani M., Sansone C. eds., 2015).

At blocks 212-218, the system generates a scaled nutrition score for the food item based on the nutrition data set and a calorie reference value. The scaled nutrition score is calculated from the nutrition data set and scaled to the calorie reference value in order to provide a quantitative metric of the aggregate nutrient content of the food item that relates to calorie intake. As will be discussed in additional detail with respect to FIG. 3, the scaled nutrition score can be represented as:

$$Scaled Nutrition Score = {\sum }_{n = 1}^{TN} PV ( n ) * CSF$$

where TN equals the total number of nutrients or nutrient subsets being assessed, PV(n) equals a nutrient point value associated with each nutrient or nutrient subset, and CSF equals a caloric scaling factor that is based on the calorie reference value (e.g., a ratio between the calorie reference value and the maximum total points). The scaled nutrition score may be positive, negative, or zero. A positive scaled nutrition score indicates that the food item is likely to have beneficial health effects when consumed, while a negative value indicates that the food item is likely to have neutral or detrimental health effects when consumed.

At block 212, the system selects a nutrient or nutrient subset to calculate a nutrition point value associated with the selected nutrient or nutrient subset. A subset can include all of the nutrients in the nutrient data set or only some of the nutrients in the nutrient data set. For example, a subset can include one, two, three, four, five, six, seven, eight, nine, ten, 11, 12, 13, 14, 15, 20, 25, 30, 40, 50, or more nutrients from the nutrient data set. Optionally, a subset can include only single category of nutrients (e.g., only vitamins, only amino acids, only minerals, etc.). Alternatively, a subset can include multiple categories of nutrients (e.g., two, three, four, or more categories). Different subsets can be entirely distinct from each other, or may have one or more nutrients in common.

At block 214, the system calculates at least one nutrition point value for the selected nutrient or nutrient subset. Each point value provides a quantitative result (e.g., a number of points) that may be positive, negative, or zero. For example, a positive value may indicate that a particular nutrient or nutrients are likely to have beneficial health effects, while a negative value may indicate that the nutrient(s) are likely to have detrimental health effects. The nutrition point value(s) associated with each nutrient or nutrient subset can be determined in many different ways. For example, the nutrition point value(s) for each nutrient or nutrient subset can be determined using any of the following calculation types: a comparison between the amount of a nutrient in the food item and a recommended intake value for the nutrient (e.g., a recommended daily intake value); a fraction or percentage of the amount of a nutrient in the food item relative to the recommended intake value; a ratio between the amounts of two or more nutrients; whether the ratio is less than, greater than, or equal to a threshold value; whether a nutrient is present or absent in the food item; whether the amount of a nutrient is less than, greater than, or equal to a threshold value; whether the amount of a first nutrient is less than, greater than, or equal to the amount of a second nutrient; whether a particular nutrient subset is present or absent in the food item; whether the total amount of the nutrient subset is less than, greater than, or equal to a threshold value; whether the total amount of a first nutrient subset is less than, greater than, or equal to the total amount of a second nutrient subset; or any suitable combination thereof. In embodiments where the nutrition subset includes a plurality of different nutrients, the system may calculate an individual point value for each nutrient, or may calculate a single point value representing the aggregated health effect of the nutrients.

At decision block 216, the system determines whether any additional nutrients or nutrient subsets remain to be assessed. If additional nutrients or nutrient subsets remain to be assessed, processing returns to block 212 where the next nutrient or nutrient subset is selected. Otherwise, processing continues to block 218. In some embodiments, the processes of blocks 212-216 are repeated multiple times to calculate nutrition point values for multiple nutrients or nutrient subsets (e.g., at least one, two, three, four, five, six, seven, eight, nine, ten, 11, 12, 13, 14, 15, 20, 25, 30, 40, 50, or more nutrients or nutrient subsets). Some or all of the nutrition point values can be calculated using different calculation types. For example, a first calculation type can be used for a first nutrient subset, a second calculation type can be used for a second nutrient subset, a third calculation type can be used for a third nutrient subset, and so on.

At block 218, the system calculates the scaled nutrition score based on the nutrient point values and a calorie reference value. In some embodiments, the scaled nutrition score is calculated by scaling each nutrient point value by the calorie reference value to generate nutrient subscores, and then summing the resulting scaled subscores. In other embodiments, the point values are first combined to generate a raw nutrition score, and the raw nutrition score is subsequently be scaled to the calorie reference value to generate the scaled nutrition score. The point values or scaled subscores can be combined with each other in various ways to produce the scaled nutrition score, e.g., by summing, weighted averages, etc. Optionally, some or all of the point values or scaled subscores may be assigned different weights when calculating the scaled nutrition score.

In some embodiments, the calorie reference value is a fixed value corresponding to an average and/or recommended daily calorie intake for an individual. The calorie reference value can be any value within a range from 1000 to 3000, such as 1200, 1400, 1600, 1800, 2000, 2200, 2400, 2600, 2800, or 3000. The calorie reference value can be determined independently of the particular user's characteristics, or can be personalized to the particular user. In some embodiments, the calorie reference value is personalized based on one or more factors that impact recommended calorie intake, such as age, gender, height, weight, BMI, physical activity level (e.g., sedentary, moderately active, active), pregnancy and/or lactation status, and/or health goals (e.g., weight loss, weight gain, weight maintenance, target calorie intake). In such embodiments, the process 200 can further include receiving user data including any of the above factors (e.g., from any of the user devices 108a-c of FIG. 1) and determining the calorie reference value based on the user data.

Optionally, the scoring methodology can be personalized to the particular user. For example, the recommended intake values for some or all of the nutrients may vary based on the particular user's characteristics (e.g., age, gender, height, weight, BMI, physical activity level, pregnancy and/or lactation status). Additionally, the user may be able to modify the scoring methodology, e.g., to account for their own health goals, dietary preferences, etc. For example, the user can indicate whether particular nutrients should be included or omitted when computing the scaled nutrition score. As another example, the user can include other factors that influence the scaled nutrition score, such as whether the food item includes artificial ingredients, whether the food item comports with certain dietary preferences or restrictions (e.g., gluten-free, vegetarian, vegan), and so on. In such embodiments, the process 200 can further include receiving user input indicating modifications to the scoring methodology (e.g., from any of the user devices 108a-c of FIG. 1) and generating the scaled nutrition score in view of the modifications.

At block 220, the system generates a nutrition quality ratio (NQR) for the food item based on the scaled nutrition score. In some embodiments, the nutrition quality ratio is calculated by dividing the scaled nutrition score by the number of calories per serving of the food item. Accordingly, the nutrition quality ratio provides a quantitative, per-calorie representation of the nutritional value of the food item. For example, a nutritional quality ratio greater than or equal to 1 may indicate that the food item is nutrient-dense, i.e., the nutrient content of the food item is greater than or equal to the calorie content of the food item. Conversely, a nutritional quality ratio less than 1 may indicate that the food item is relatively nutrient-sparse, i.e., the nutrient content of the food item is less than the calorie content of the food item. The nutrition quality ratio may be positive, negative, or zero. A positive ratio indicates that the food item is likely to have beneficial health effects when consumed, while a negative ratio indicates that the food item is likely to have neutral or detrimental health effects when consumed. In other embodiments, however, block 220 is optional and can be omitted.

Optionally, at block 222, the system further generates and outputs a recommendation based on the scaled nutrition score and/or the nutrition quality ratio. The recommendation can inform a user of an action they should take with respect to the food item. For example, the recommendation can inform the user whether the food item is healthy, moderately healthy, moderately unhealthy, or unhealthy. As another example, the recommendation can instruct the user to consume the food item, consume with caution (e.g., consume in limited amounts), or not to consume the food item. In a further example, the recommendation can provide other types of information related to food and health, e.g., comparing the food item to other food items, predicting how consumption of the food item would impact the user's health goals, suggesting other food items the user should consume with the food item or as an alternative to the food item, alerting the user to potential issues with their diet (e.g., not enough fiber, too much sugar), etc.

In some embodiments, the recommendation can involve assigning the food item to a health category based on the value of the nutrition quality ratio, e.g., as indicated in Table 1 below. Optionally, each health category is associated with a color code to be displayed with the recommendation so as to provide a clear and distinctive visual indicator for the user. The health categories, threshold values, and color codes shown in Table 1 are provided merely as an example and can be modified in other embodiments.

TABLE 1
Exemplary Health Categories for Food Items.

| Category and Color Code | Value of Nutrient Quality Ratio (NQR) |
|---|---|
| Healthy (Green) | NQR > 1 |
| Moderately healthy (Yellow) | 0.5 < NQR ≤ 1 |
| Moderately unhealthy (Red) | 0 < NQR ≤ 0.5 |
| Unhealthy (Black) | NQR ≤ 0 |

The process 200 can be performed in response to a request from a user (e.g., any of the users 110a-c of FIG. 1) or from food manufacturers or distributors 112 for purposes of product labeling and advertising. For example, the user may request a nutritional evaluation of one or more food items, e.g., when deciding whether to purchase or consume the food item, when planning a future meal, when assessing the health impact of previous meals, etc. The request can include an identification of one or more food items to be evaluated, and, optionally, other input data used to perform the evaluation (e.g., user data, modifications to the scoring methodology, etc.). The user can input the request via a suitable graphical user interface displayed on a user device (e.g., any of the user devices 108a-c of FIG. 1), and the user device can transmit the request to the system. Subsequently, the system generates the evaluation results as discussed above, and transmits the results to the user device for display via the graphical user interface. In other embodiments, however, the evaluation results are generated independently of any user request and stored in a database (e.g., database 104 of FIG. 1). When the system receives a request from the user or from food manufacturers or distributors 112, the system simply retrieves and outputs the appropriate results (e.g., via a graphical user interface displayed on a user device).

In some embodiments, some or all of the steps of the process 200 are repeated multiple times to generate evaluation results (e.g., scaled nutrition scores, nutrition quality ratios and/or recommendations) for a plurality of different food items (e.g., at least 10, 100, or 1000 different food items). Different food items may have highly diverse compositions (e.g., different types and amounts of ingredients and nutrients, different calorie densities) such that the analysis becomes very complex and may require processing large amounts of food/nutrient data from many different data sources. Additionally, some or all of the steps of the process 200 may be repeated periodically to update the evaluation results, for example, if new food/nutrient data becomes available and/or if there are changes in the scoring methodology (e.g., based on research, clinical results, etc.). Accordingly, the nutrition evaluation techniques described herein are not suitable for implementation using manual processes.

FIG. 3 is a flow diagram illustrating an exemplary process 300 performed by the system for generating a scaled nutrition score. The process 300 may be performed as part of the process 200 of FIG. 2 (e.g., as a sub-process of blocks 212-218). The process 300 can be performed by any embodiment of the nutrition evaluation system and associated devices described herein. For example, the process 300 can be performed entirely by the servers 102 of FIG. 1, entirely by one or more of the user devices 108a-c of FIG. 1, or by a combination of the servers 102 and user devices 108a-c.

Three different types of calculations associated with nutrients or nutrient subsets are contemplated in process 300. The first relates to the calculation of subscores for individual nutrients based on the daily recommended intake value for those nutrients, the second relates to the calculation of subscores for various ratios of nutrients that are believed to be beneficial or detrimental, and the third relates to the calculation of subscores for certain nutrients based on the comparison of those nutrients with certain threshold values that are believed to be beneficial or detrimental. Each subscore calculation is discussed in turn.

The process 300 begins at block 310 where the system calculates first subscores by comparing a first subset of nutrients to recommended daily intake values for those nutrients. The first subset can include nutrients from a plurality of different categories, e.g., at least one vitamin, at least one mineral, at least one fatty acid, and/or fiber. In some embodiments, the first subset includes one or more the following nutrients: vitamin A, vitamin B1, vitamin B2, vitamin B3, vitamin B5, vitamin B6, vitamin B7, vitamin B9, vitamin B9, vitamin B12, vitamin C, vitamin D, vitamin E, vitamin K, choline, calcium, chromium, copper, iodine, iron, magnesium, manganese, molybdenum, phosphorus, potassium, selenium, zinc, chloride, histidine, isoleucine, leucine, lysine, methionine, cysteine, phenylalanine, tyrosine, threonine, tryptophan, valine, omega-3 fatty acid, omega-6 fatty acid, fiber, or phytonutrients.

For each nutrient, an unscaled point value p can be calculated as follows:

$$if \frac{x}{D} < 1 , p = \frac{x}{D} \times {p}_{max}$$

$$if \frac{x}{D} \geq 1 , p = {p}_{max}$$

where x is the amount of the nutrient per serving of the food item, D is the recommended daily intake of the nutrient, and pₘₐₓ is the maximum point value for the nutrient. Thus, a nutrient is awarded the full point value if the amount of the nutrient per serving of the food item is greater than or equal to the recommended daily intake, and earns a fractional point value if the amount is less than the recommended daily intake. In some embodiments, the maximum point value pₘₐₓ is the same for all nutrients in the first subset such that each nutrient is weighted equally in calculating the first score. In other embodiments, pₘₐₓ may be different for different nutrients such that some nutrients are weighted more heavily than others. For example, pₘₐₓ can be 2 for fiber and phytonutrients, and 1 for all other nutrients (e.g., vitamins, minerals, amino acids, fatty acids).

A scaled subscore s for each nutrient can be calculated from the unscaled point value p as follows:

$$s = \frac{p}{T} \times C$$

where T is the total number of unscaled points possible from the entire calculation, and C is the calorie reference value. The ratio C/T corresponds to the caloric scaling factor (CSF) for the scoring methodology.

Table 2 provides examples of recommended daily intake values for various nutrients that may be used to calculate the raw point value p for the first subset of nutrients. The values shown in Table 2 are determined for two reference individuals, a generic male (e.g., an 80 kg male with a 2000 calorie diet) and a generic female (e.g., a 59 kg female with a 1500 calorie diet). In other embodiments, however, the recommended daily intake values can be determined for reference individuals having different characteristics (e.g., different gender, weight, age, dietary intake, etc.) or can be personalized to a particular user's characteristics.

TABLE 2
Exemplary Recommended Daily Intake Values.

| | Recommended | | Recommended | |
|---|---|---|---|---|
| | Daily Intake | | Daily Intake | |
| Nutrient | (Generic Male) | | (Generic Female) | |
| Vitamin A | 900 | μg RAE | 700 | μg RAE |
| Vitamin B1 (thiamin) | 1.2 | mg | 1.1 | mg |
| Vitamin B2 (riboflavin) | 1.3 | mg | 1.1 | mg |
| Vitamin B3 (niacin) | 16 | mg NE | 14 | mg NE |
| Vitamin B5 (pantothenic acid) | 5 | mg | 5 | mg |
| Vitamin B6 (pyridoxine) | 1.3 | mg | 1.3 | mg |
| Vitamin B7 (biotin) | 30 | μg | 30 | μg |
| Vitamin B9 (folic acid) | 400 | μg DFE | 400 | μg DFE |
| Vitamin B12 (cobalamin) | 2.4 | μg | 2.4 | μg |
| Vitamin C (ascorbic acid) | 90 | mg | 75 | mg |
| Vitamin D (vitamin | 15 | μg | 15 | μg |
| D2/ergocalciferol, | | | | |
| vitamin D3/cholecalciferol) | | | | |
| Vitamin E (tocopherol) | 15 | mg | 15 | mg |
| Vitamin K (phylloquinone) | 120 | μg | 90 | μg |
| Choline (vitamin Bp) | 550 | mg | 425 | mg |
| Calcium | 1000 | mg | 1000 | mg |
| Chloride | 2.3 | g | 2.3 | g |
| Chromium | 35 | μg | 25 | μg |
| Copper | 900 | μg | 900 | μg |
| Iodine | 150 | μg | 150 | μg |
| Iron | 8 | mg | 18 | mg |
| Magnesium | 400 | mg | 310 | mg |
| Manganese | 2.3 | mg | 1.8 | mg |
| Molybdenum | 45 | μg | 45 | μg |
| Phosphorus | 700 | mg | 700 | mg |
| Potassium | 3400 | mg | 2600 | mg |
| Selenium | 55 | μg | 55 | μg |
| Zinc | 11 | mg | 8 | mg |
| Sodium | 1500 | mg | 1500 | mg |
| Histidine | 1120 | mg | 827 | mg |
| Isoleucine | 1520 | mg | 1123 | mg |
| Leucine | 3360 | mg | 2482 | mg |
| Lysine | 3040 | mg | 2246 | mg |
| Methionine + Cysteine SAA | 1520 | mg | 1123 | mg |
| Phenylalanine + Tyrosine | 2640 | mg | 1950 | mg |
| Threonine | 1600 | mg | 1182 | mg |
| Tryptophan | 400 | mg | 296 | mg |
| Valine | 320 | mg | 236 | mg |
| Omega-3 (alpha-linolenic acid) | 1.6 | g | 1.2 | g |
| Omega-6 (linoleic acid) | 17 | g | 12 | g |
| Fiber | 38 | g | 25 | g |
| Sugar | 50 | g | 50 | g |
| Phytonutrients | 150 | mg | 150 | mg |

At block 320, the system calculates second subscores by computing ratios between a second subset of nutrients. For example, in some embodiments, a subscore is determined by calculating a potassium to sodium ratio (K/Na ratio) and/or a subscore is determined by calculating an omega-6 fatty acid to omega-3 fatty acid ratio (omega-6/omega-3 ratio). Each ratio is compared to a threshold value to determine the points awarded for that ratio. The threshold value can be calculated based on the recommended daily intake values for the nutrients in the ratio.

For example, for a ratio between two nutrients x₁, x₂, if the threshold value is a minimum value tₘᵢₙ for the ratio (i.e., a larger ratio is more favorable), the unscaled point value p can be calculated as follows:

$$if \frac{{x}_{1}}{{x}_{2}} \geq {t}_{mi n} , p = {p}_{max}$$

$$if \frac{{x}_{1}}{{x}_{2}} < {t}_{min} , p = \frac{{x}_{2}}{{x}_{1}} \times {p}_{max}$$

$$if {x}_{1} = {x}_{2} = 0 , p = 0$$

Thus, the full point value is awarded if the nutrient ratio is greater than or equal to the threshold value, a fractional point value is awarded if the nutrient ratio is less than the threshold value, and no points are awarded if the amount of both nutrients is zero. For example, in some embodiments, the K/Na ratio is scored based on a minimum threshold value tₘᵢₙ of 2 (e.g., for a generic male) or 1.73 (e.g., for a generic female), and a maximum point value pₘₐₓ of 1.

Conversely, if the threshold value is a maximum value tₘₐₓ for the ratio (i.e., a smaller ratio is more favorable), the unscaled point value p can be calculated as follows:

$$if \frac{{x}_{1}}{{x}_{2}} \leq {t}_{max} , p = {p}_{max}$$

$$if \frac{{x}_{1}}{{x}_{2}} > {t}_{max} , p = \frac{{x}_{2}}{{x}_{1}} \times {p}_{max}$$

$$if {x}_{1} = {x}_{2} = 0 , p = 0$$

Thus, the full point value is awarded if the nutrient ratio is less than or equal to the threshold value, a fractional point value is awarded if the nutrient ratio is greater than the threshold value, and no points are awarded if the amount of both nutrients is zero. For example, in some embodiments, the omega-6/omega-3 ratio is scored based on a maximum threshold value tₘₐₓ of 10.625 (e.g., for a generic male) or 10.909 (e.g., for a generic female), and a maximum point value pₘₐₓ of 1.

A scaled subscore s for each nutrient ratio can be calculated from each of the unscaled point values p as follows:

$$s = \frac{p}{T} \times C$$

where T is the total number of unscaled points possible and C is the calorie reference value.

At block 330 the system calculates third subscores by comparing a third subset of nutrients to one or more threshold values. For each nutrient, the point value awarded depends on whether the amount of the nutrient is greater than, equal to, or less than one or more threshold values. The point value can be positive, negative, or zero, depending on the recommended daily intake for the nutrient and/or the expected health impact of consuming too much or too little of the nutrient.

For example, an unscaled point value p can be calculated based on the amount of added sugar per serving of the food item x_{sugar} as follows:
if x_{sugar}≤5 g, p=1
if 5 g<x_{sugar}≤10 g, p=0.5
if 10 g<x_{sugar}≤15 g, p=−1
if 15 g<x_{sugar}≤20 g, p=−5
if x_{sugar}>20 g, p=−10
   Thus, the unscaled point value p for sugar is positive if the amount of added sugar is sufficiently low, and is negative if the amount of added sugar is too high. In some embodiments, the possibility of negative point values for added sugar reflects the numerous health risks associated with excessive sugar consumption (e.g., weight gain, obesity, Type 2 diabetes, high triglycerides, high cholesterol, hypertension, stroke, coronary heart disease, cancer, and tooth decay). The threshold values for the sugar scoring methodology can be determined based on the recommended daily intake for sugar (e.g., 5%, 10%, 20%, 30%, 40%, 50% of the recommended daily intake). In some embodiments, the full point value is awarded if the amount of added sugar is less than or equal to 10% of the recommended daily intake; a fractional point value is awarded if the amount of added sugar is between 10% and 20% of the recommended daily intake; and negative point values are awarded if the amount of added sugar is greater than 20% of the recommended daily intake.

As another example, an unscaled point value p can be calculated based on the amount of sodium per serving of the food item x_{Na} as follows:
if x_{Na}≤100 mg, p=0
if 100 mg<x_{Na}≤200 mg, p=0.5
if 200 mg<x_{Na}≤400 mg, p=1
if 400 mg<x_{Na}≤600 mg, p=0.5
if 600 mg<x_{Na}≤800 mg, p=0
if 800 mg>x_{Na}≤x_{Na}≤1000 mg, p=−2
if x_{Na}>1000 mg, p=−5
   Thus, the unscaled point value p for sodium is positive if the amount of sodium is within a predetermined range (e.g., 100 mg to 600 mg), and is negative if the amount of sodium is above a threshold judged to be too high (greater than 800 mg). The threshold values for the sodium calculation can be determined based on the recommend daily intake for sodium and/or other considerations. For example, the possibility of negative point values for high amounts of sodium can reflect the health risks associated with excessive sodium consumption (e.g., hypertension, kidney disease, heart failure, and stroke).

A scaled subscore s can be calculated for each analyzed nutrient from each of the unscaled point values p as follows:

$$s = \frac{p}{T} \times C$$

where T is total number of unscaled points possible and C is the calorie reference value.

At block 340, a scaled nutrition score (SNS) is generated by combining the subscores calculated in blocks 310-330. For example, the scaled nutrition score can simply be the sum of all calculated subscores:

SNS=Σsᵢ

where sᵢ is the i^{th} scaled subscore. In other embodiments, however, the scaled nutrition score can be calculated from the subscores using different approaches (e.g., weighted sum, weighted average, etc.).

The nutrition quality ratio (NQR) is then generated by dividing the scaled nutrition score by the number of calories per serving of the food item c:

$$NQR = \frac{SNS}{c}$$

Table 3 provides exemplary maximum unscaled point values and scaled subscores that may be used in the calculations described above in connection with the process 300. The scaled subscores in Table 3 are scaled to reference calorie values of 2000 or 1500.

TABLE 3
Exemplary Maximum Unsealed Point Values and Scaled Subscores

| | | Maximum | Maximum |
|---|---|---|---|
| | Maximum | Scaled | Scaled |
| | Unscaled | Subscore | Subscore |
| | Point Value | (C = 2000) | (C = 1500) |
| Vitamin A | 1 | 45.5 | 34.1 |
| Vitamin B1 | 1 | 45.5 | 34.1 |
| Vitamin B2 | 1 | 45.5 | 34.1 |
| Vitamin B3 | 1 | 45.5 | 34.1 |
| Vitamin B5 | 1 | 45.5 | 34.1 |
| Vitamin B6 | 1 | 45.5 | 34.1 |
| Vitamin B7 | 1 | 45.5 | 34.1 |
| Vitamin B9 | 1 | 45.5 | 34.1 |
| Vitamin B12 | 1 | 45.5 | 34.1 |
| Vitamin C | 1 | 45.5 | 34.1 |
| Vitamin D | 1 | 45.5 | 34.1 |
| Vitamin E | 1 | 45.5 | 34.1 |
| Vitamin K | 1 | 45.5 | 34.1 |
| Choline | 1 | 45.5 | 34.1 |
| Calcium | 1 | 45.5 | 34.1 |
| Chloride | 1 | 45.5 | 34.1 |
| Chromium | 1 | 45.5 | 34.1 |
| Copper | 1 | 45.5 | 34.1 |
| Iodine | 1 | 45.5 | 34.1 |
| Iron | 1 | 45.5 | 34.1 |
| Magnesium | 1 | 45.5 | 34.1 |
| Manganese | 1 | 45.5 | 34.1 |
| Molybdenum | 1 | 45.5 | 34.1 |
| Phosphorus | 1 | 45.5 | 34.1 |
| Potassium | 1 | 45.5 | 34.1 |
| Selenium | 1 | 45.5 | 34.1 |
| Zinc | 1 | 45.5 | 34.1 |
| Histidine | 1 | 45.5 | 34.1 |
| Isoleucine | 1 | 45.5 | 34.1 |
| Leucine | 1 | 45.5 | 34.1 |
| Lysine | 1 | 45.5 | 34.1 |
| Methionine + Cysteine SAA | 1 | 45.5 | 34.1 |
| Phenylalanine + Tyrosine | 1 | 45.5 | 34.1 |
| Threonine | 1 | 45.5 | 34.1 |
| Tryptophan | 1 | 45.5 | 34.1 |
| Valine | 1 | 45.5 | 34.1 |
| Omega-3 | 1 | 45.5 | 34.1 |
| Omega-6 | 1 | 45.5 | 34.1 |
| Fiber | 2 | 90.9 | 68.2 |
| K/Na Ratio | 1 | 45.5 | 34.1 |
| Omega-6/Omega-3 Ratio | 1 | 45.5 | 34.1 |
| Sugar | 1 | 45.5 | 34.1 |
| Sodium | 1 | 45.5 | 34.1 |
| Total Possible Points | 44 | 2000 | 1500 |

Tables 4-7 provide exemplary nutrition evaluation results for three different food items (nutrient chocolate shake, white rice, soda) performed in accordance with the process 300. The calculations for Tables 4-6 were performed for a generic male and a calorie reference value of 2000 using the recommended daily intake values of Table 2 and the maximum unscaled point values and scaled subscores of Table 3. The calculations for Tables 7 were performed for a generic female and a calorie reference value of 1500 using the recommended daily intake values of Table 2 and the maximum unscaled point values and scaled subscores of Table 3. Each Table lists the nutrients used in the calculation, the amount of each nutrient per serving of the food item, and the scaled subscore awarded for the nutrient, separated by calculation type. Each Table also provides the final scaled nutrition score and nutrient quality ratio.

TABLE 4
Exemplary Nutrition Evaluation of a Nutrient
Chocolate Shake (Generic Male)

| Nutrient | Amount Per Serving | | Scaled Subscore |
|---|---|---|---|
| First Calculation Type | | | |
| Vitamin A | 536.4 | μg RAE | 27.1 |
| Vitamin B1 | 0.54 | mg | 20.6 |
| Vitamin B2 | 0.66 | mg | 22.9 |
| Vitamin B3 | 6.87 | mg NE | 19.5 |
| Vitamin B5 | 3.57 | mg | 32.5 |
| Vitamin B6 | 0.69 | mg | 24.1 |
| Vitamin B7 | 99 | μg | 45.5 |
| Vitamin B9 | 227.52 | μg DFE | 25.9 |
| Vitamin B12 | 2.14 | μg | 40.5 |
| Vitamin C | 30.06 | mg | 15.2 |
| Vitamin D | 7.12 | μg | 21.6 |
| Vitamin E | 5.02 | mg | 15.2 |
| Vitamin K | 45.31 | mg | 17.2 |
| Choline | 188.3 | mg | 15.6 |
| Calcium | 577.9 | mg | 26.3 |
| Chloride | 2.3 | g | 44.9 |
| Chromium | 40 | μg | 45.5 |
| Copper | 662 | μg | 33.4 |
| Iodine | 49.5 | μg | 15.0 |
| Iron | 5.5 | mg | 31.2 |
| Magnesium | 190.7 | mg | 21.7 |
| Manganese | 0.8 | mg | 15.0 |
| Molybdenum | 24.8 | μg | 25.0 |
| Phosphorus | 585.9 | mg | 38.0 |
| Potassium | 1948.4 | mg | 26.0 |
| Selenium | 24.2 | μg | 20.0 |
| Zinc | 5.6 | mg | 23.2 |
| Histidine | 496.64 | mg | 20.2 |
| Isoleucine | 1329.7 | mg | 39.8 |
| Leucine | 2507.5 | mg | 33.9 |
| Lysine | 2110.9 | mg | 31.6 |
| Methionine + Cysteine SAA | 990.0 | mg | 29.6 |
| Phenylalanine + Tyrosine | 1523.6 | mg | 26.2 |
| Threonine | 1395.8 | mg | 39.7 |
| Tryptophan | 348.2 | mg | 39.6 |
| Valine | 1468.8 | mg | 45.5 |
| Omega-3 | 0.6 | g | 18.2 |
| Omega-6 | 1.5 | g | 4.0 |
| Fiber | 5.8 | g | 13.8 |
| Second Calculation Type | | | |
| K/Na | 25.2 | | 45.5 |
| Omega-6/Omega-3 | 2.4 | | 45.5 |
| Third Calculation Type | | | |
| Sugar | 5.0 | g | 45.5 |
| Sodium | 77.3 | mg | 0 |

| | |
|---|---|
| Scaled Nutrition Score | 1187.1 |
| Calories per Serving | 240 |
| Nutrition Quality Ratio | 4.95 |

TABLE 5
Exemplary Nutrition Evaluation of White Rice (Generic Male)

| Nutrient | Amount Per Serving | | Scaled Subscore |
|---|---|---|---|
| First Calculation Type | | | |
| Vitamin A | 0 | μg RAE | 0 |
| Vitamin B1 | 0.04 | mg | 1.33 |
| Vitamin B2 | 0.02 | mg | 0.80 |
| Vitamin B3 | 0.51 | mg NE | 1.43 |
| Vitamin B5 | 0.37 | mg | 3.40 |
| Vitamin B6 | 0.05 | mg | 1.57 |
| Vitamin B7 | 0 | μg | 0 |
| Vitamin B9 | 1.74 | μg DFE | 0.20 |
| Vitamin B12 | 0 | μg | 0 |
| Vitamin C | 0 | mg | 0 |
| Vitamin D | 0 | μg | 0 |
| Vitamin E | 0.07 | mg | 0.21 |
| Vitamin K | 0 | μg | 0 |
| Choline | 3.65 | mg | 0.30 |
| Calcium | 3.48 | | 0.16 |
| Chloride | 0 | g | 0 |
| Chromium | 0 | μg | 0 |
| Copper | 0.09 | μg | 0 |
| Iodine | 0 | μg | 0 |
| Iron | 0.24 | mg | 1.39 |
| Magnesium | 8.70 | mg | 0.99 |
| Manganese | 0.46 | mg | 9.01 |
| Molybdenum | 0 | μg | 0 |
| Phosphorus | 13.90 | mg | 0.90 |
| Potassium | 17.40 | mg | 0.23 |
| Selenium | 9.74 | μg | 8.05 |
| Zinc | 0.71 | mg | 2.95 |
| Histidine | 82 | mg | 3.4 |
| Isoleucine | 151 | mg | 4.6 |
| Leucine | 291 | mg | 4.0 |
| Lysine | 127 | mg | 1.9 |
| Methionine + Cysteine SAA | 153 | mg | 4.7 |
| Phenylalanine + Tyrosine | 305 | mg | 5.3 |
| Threonine | 125 | mg | 3.6 |
| Tryptophan | 40 | mg | 4.6 |
| Valine | 214 | mg | 30.9 |
| Omega-3 | 0 | g | 0 |
| Omega-6 | 0 | g | 0 |
| Fiber | 1.74 | g | 4.16 |
| Second Calculation Type | | | |
| K/Na | 0 | | 0 |
| Omega-6/Omega-3 | 0 | | 0 |
| Third Calculation Type | | | |
| Sugar | 0 | g | 45.45 |
| Sodium | 8.70 | mg | 0 |

| | |
|---|---|
| Scaled Nutrition Score | 145.6 |
| Calories per Serving | 169 |
| Nutrition Quality Ratio | 0.86 |

TABLE 6
Exemplary Nutrition Evaluation of Soda (Generic Male)

| Nutrient | Amount Per Serving | | Scaled Subscore |
|---|---|---|---|
| First Calculation Type | | | |
| Vitamin A | 0 | μg RAE | 0 |
| Vitamin B1 | 0 | mg | 0 |
| Vitamin B2 | 0 | mg | 0 |
| Vitamin B3 | 0 | mg NE | 0 |
| Vitamin B5 | 0 | mg | 0 |
| Vitamin B6 | 0 | mg | 0 |
| Vitamin B7 | 0 | μg | 0 |
| Vitamin B9 | 0 | μg DFE | 0 |
| Vitamin B12 | 0 | μg | 0 |
| Vitamin C | 0 | mg | 0 |
| Vitamin D | 0 | μg | 0 |
| Vitamin E | 0 | mg | 0 |
| Vitamin K | 0 | μg | 0 |
| Choline | 1.12 | mg | 0.09 |
| Calcium | 3.72 | mg | 0.17 |
| Chloride | 0 | g | 0 |
| Chromium | 0 | μg | 0 |
| Copper | 0.03 | μg | 0 |
| Iodine | 0 | μg | 0 |
| Iron | 0.07 | mg | 0.42 |
| Magnesium | 0 | mg | 0 |
| Manganese | 0 | mg | 0 |
| Molybdenum | 0 | μg | 0 |
| Phosphorus | 33.50 | mg | 2.18 |
| Potassium | 18.60 | mg | 0.25 |
| Selenium | 0.37 | μg | 0.31 |
| Zinc | 0.34 | mg | 1.38 |
| Histidine | 0 | mg | 0 |
| Isoleucine | 0 | mg | 0 |
| Leucine | 0 | mg | 0 |
| Lysine | 0 | mg | 0 |
| Methionine + Cysteine SAA | 0 | mg | 0 |
| Phenylalanine + Tyrosine | 0 | mg | 0 |
| Threonine | 0 | mg | 0 |
| Tryptophan | 0 | mg | 0 |
| Valine | 0 | mg | 0 |
| Omega-3 | 0 | g | 0 |
| Omega-6 | 0 | g | 0 |
| Fiber | 0 | g | 0 |
| Second Calculation Type | | | |
| K/Na | 0 | | 0 |
| Omega-6/Omega-3 | 0 | | 0 |
| Third Calculation Type | | | |
| Sugar | 37 | g | −454.55 |
| Sodium | 11.2 | mg | 0 |

| | |
|---|---|
| Scaled Nutrition Score | −449.8 |
| Calories per Serving | 156 |
| Nutrition Quality Ratio | −2.88 |

TABLE 7
Exemplary Nutrition Evaluation of a Nutrient
Chocolate Shake (Generic Female)

| Nutrient | Amount Per Serving | | Scaled Subscore |
|---|---|---|---|
| First Calculation Type | | | |
| Vitamin A | 536.4 | μg RAE | 26.1 |
| Vitamin B1 | 0.54 | mg | 16.9 |
| Vitamin B2 | 0.66 | mg | 20.3 |
| Vitamin B3 | 6.87 | mg NE | 16.7 |
| Vitamin B5 | 3.57 | mg | 24.3 |
| Vitamin B6 | 0.69 | mg | 18.1 |
| Vitamin B7 | 99 | μg | 34.1 |
| Vitamin B9 | 227.52 | μg DFE | 19.4 |
| Vitamin B12 | 2.14 | μg | 30.4 |
| Vitamin C | 30.06 | mg | 13.7 |
| Vitamin D | 7.12 | μg | 16.2 |
| Vitamin E | 5.02 | mg | 11.4 |
| Vitamin K | 45.31 | mg | 17.2 |
| Choline | 188.3 | mg | 15.1 |
| Calcium | 577.9 | mg | 19.7 |
| Chloride | 2.3 | g | 33.7 |
| Chromium | 40 | μg | 34.1 |
| Copper | 662 | μg | 25.1 |
| Iodine | 49.5 | μg | 11.3 |
| Iron | 5.5 | mg | 10.4 |
| Magnesium | 190.7 | mg | 21.0 |
| Manganese | 0.8 | mg | 14.4 |
| Molybdenum | 24.8 | μg | 18.8 |
| Phosphorus | 585.9 | mg | 28.5 |
| Potassium | 1948.4 | mg | 25.5 |
| Selenium | 24.2 | μg | 15.0 |
| Zinc | 5.6 | mg | 24.0 |
| Histidine | 496.64 | mg | 20.5 |
| Isoleucine | 1329.7 | mg | 34.1 |
| Leucine | 2507.5 | mg | 34.1 |
| Lysine | 2110.9 | mg | 32.0 |
| Methionine + Cysteine SAA | 990.0 | mg | 30.1 |
| Phenylalanine + Tyrosine | 1523.6 | mg | 26.6 |
| Threonine | 1395.8 | mg | 34.1 |
| Tryptophan | 348.2 | mg | 34.1 |
| Valine | 1468.8 | mg | 34.1 |
| Omega-3 | 0.6 | g | 19.8 |
| Omega-6 | 1.5 | g | 4.3 |
| Fiber | 5.8 | g | 15.8 |
| Second Calculation Type | | | |
| K/Na | 25.2 | | 34.1 |
| Omega-6/Omega-3 | 2.4 | | 34.1 |
| Third Calculation Type | | | |
| Sugar | 5.0 | g | 34.1 |
| Sodium | 77.3 | mg | 0 |

| | |
|---|---|
| Scaled Nutrition Score | 983.1 |
| Calories per Serving | 240 |
| Nutrition Quality Ratio | 4.09 |

Table 8 illustrates exemplary scaled nutrition scores, nutrition quality ratios, and health categories calculated for various food items in accordance with the process 300 of FIG. 3. The results in Table 8 were calculated for a generic male and a calorie reference value of 2000, using the recommended daily intake values of Table 2 and the maximum unscaled point values and scaled subscores of Table 3.

TABLE 8
Exemplary Nutrition Evaluation Results

| | Scaled | Nutrition | |
|---|---|---|---|
| | Nutrition | Quality | |
| Food Item | Score | Ratio | Health Category |
| Kale | 121 | 16.4 | Healthy |
| Beef Liver | 869 | 5.7 | Healthy |
| Nutrient Chocolate Shake | 1187 | 5.0 | Healthy |
| Hummus | 234 | 1.3 | Healthy |
| White Rice | 146 | 0.9 | Moderately Healthy |
| Granola | 320 | 0.5 | Moderately Unhealthy |
| Olive Oil | 65 | 0.3 | Moderately Unhealthy |
| Red Wine | 92 | 0.3 | Moderately Unhealthy |
| Onion Rings | 96 | 0.2 | Moderately Unhealthy |
| Glazed Donut | −66 | −0.2 | Unhealthy |
| Vanilla Ice Cream | −143 | −0.3 | Unhealthy |
| Soda | −450 | −2.9 | Unhealthy |

The scoring methodology described above with respect to FIG. 3 is provided as an example and can be modified in many different ways. In other embodiments, for example, one or more of blocks 310, 320, or 330 can be omitted, such that the scaled nutrition score is calculated from only the first type of subscore computation, only the second type of subscore computation, only the third type of subscore computations, only the first and second types, etc. The nutrients included in the first, second, and third types of computations can also be varied as desired. For example, a subscore can be calculated based on the amount of phytonutrients in the food item with pₘₐₓ=2. As another example, a subscore can be calculated based on a ratio between saturated fat to polyunsaturated fat. Additionally, the point values and threshold values used in the above calculations are exemplary values and can be modified as desired.

The above Detailed Description of examples of the disclosed technology is not intended to be exhaustive or to limit the disclosed technology to the precise form disclosed above. While specific examples for the disclosed technology are described above for illustrative purposes, various equivalent modifications are possible within the scope of the disclosed technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further, any specific numbers noted herein are only examples: alternative implementations may employ differing values or ranges.

For purposes of this description, a “food item” includes both processed and unprocessed foods and beverages. A “nutrient” as described herein includes any substance that supports physiological functions (e.g., metabolism, growth, tissue repair, and reproduction) and includes both essential nutrients (i.e., nutrients that cannot be synthesized in the body in sufficient quantities for normal physiological function) and nonessential nutrients (i.e., nutrients that can be synthesized by the body in sufficient quantities for normal physiological function and/or are not required but have an impact on normal physiological function).

While the above description describes certain examples of the disclosed technology, and describes the best mode contemplated, no matter how detailed the above appears in text, the disclosed technology can be practiced in many ways. Details of the system and method may vary considerably in their specific implementations, while still being encompassed by the technology disclosed herein. As noted above, particular terminology used when describing certain features or aspects of the disclosed technology should not be taken to imply that the terminology is being redefined herein to be restricted to any specific characteristics, features, or aspects of the disclosed technology with which that terminology is associated. In general, the terms used in the following claims should not be construed to limit the disclosed technology to the specific examples disclosed in the specification, unless the above Detailed Description section explicitly defines such terms.

I/We claim:

1. A computer-implemented method for evaluating nutrient content of a food item, the method comprising:
maintaining a nutrient data set for the food item, the nutrient data set including a plurality of nutrients in the food item and an amount of each nutrient per serving of the food item;
determining a first plurality of subscores using a first subset of the nutrients by comparing the amount of each nutrient of the first subset to a recommended intake value for the nutrient;
determining a second plurality of subscores using a second subset of the nutrients by calculating at least one ratio between the amounts of the nutrients of the second subset;
determining a third plurality of subscores using a third subset of the nutrients by comparing the amount of each nutrient of the third subset to one or more respective threshold values;
generating a scaled nutrition score by combining the first, second, and third pluralities of subscores, wherein the scaled nutrition score is scaled to a calorie reference value; and
generating a nutrition quality ratio by dividing the scaled nutrition score by a number of calories per serving of the food item.

2. The computer-implemented method of claim 1, wherein the calorie reference value is 2000.

3. The computer-implemented method of claim 1, wherein the calorie reference value is a fixed value.

4. The computer-implemented method of claim 1, wherein the calorie reference value is personalized based on user data.

5. The computer-implemented method of claim 1, wherein generating the scaled nutrition score comprises scaling each of the first, second, and third pluralities of subscores to the calorie reference value.

6. The computer-implemented method of claim 1, wherein generating the scaled nutrition score comprises:
combining the first, second, and third pluralities of subscores to generate a combined score; and
scaling the combined score to the calorie reference value.

7. The computer-implemented method of claim 1, wherein the plurality of nutrients includes one or more of the following: a vitamin, a mineral, an amino acid, a fatty acid, a phytonutrient, fiber, or sugar.

8. The computer-implemented method of claim 1, wherein the plurality of nutrients includes one or more of the following: vitamin A, vitamin B1, vitamin B2, vitamin B3, vitamin B5, vitamin B6, vitamin B7, vitamin B9, vitamin B9, vitamin B12, vitamin C, vitamin D, vitamin E, vitamin K, choline, calcium, chromium, copper, iodine, iron, magnesium, manganese, molybdenum, phosphorus, potassium, selenium, zinc, chloride, sodium, histidine, isoleucine, leucine, lysine, methionine, cysteine, phenylalanine, tyrosine, threonine, tryptophan, valine, omega-3 fatty acid, alpha-linolenic acid, omega-6 fatty acid, linoleic acid, fiber, sugar, flavonoids, carotenoids, or polyphenols.

9. The computer-implemented method of claim 1, wherein the first subset of nutrients includes at least one vitamin, at least one mineral, at least one fatty acid, at least one phytonutrient, and fiber.

10. The computer-implemented method of claim 1, wherein the second subset of nutrients includes potassium and sodium, and the at least one ratio includes a potassium to sodium ratio.

11. The computer-implemented method of claim 1, wherein the second subset of nutrients includes an omega-3 fatty acid and an omega-6 fatty acid, and the at least one ratio includes an omega-6 fatty acid to omega-3 fatty acid ratio.

12. The computer-implemented method of claim 1, wherein determining the second plurality of subscores further comprises comparing the at least one ratio to a threshold value.

13. The computer-implemented method of claim 1, wherein the third subset of nutrients includes sugar.

14. The computer-implemented method of claim 13, wherein determining the third plurality of subscores further comprises assigning a negative point value if the amount of sugar exceeds a threshold value.

15. The computer-implemented method of claim 13, wherein determining the third plurality of subscores further comprises:
assigning a positive point value if the amount of sugar is less than a first threshold value; and
assigning a negative point value if the amount of sugar exceeds a second threshold value.

16. The computer-implemented method of claim 15, wherein the negative point value is a first negative point value, and wherein determining the third plurality of subscores further comprises assigning a second negative point value if the sugar exceeds a third threshold value.

17. A non-transitory computer-readable medium containing instructions configured to cause one or more processors to perform a method for evaluating nutrient content of a food item, the method comprising:
maintaining a nutrient data set for the food item, the nutrient data set including a plurality of nutrients in the food item and an amount of each nutrient per serving of the food item;
determining a first plurality of subscores using a first subset of the nutrients by comparing the amount of each nutrient of the first subset to a recommended intake value for the nutrient;
determining a second plurality of subscores using a second subset of the nutrients by calculating at least one ratio between the amounts of the nutrients of the second subset;
determining a third plurality of subscores using a third subset of the nutrients by comparing the amount of each nutrient of the third subset to one or more respective threshold values;
generating a scaled nutrition score by combining the first, second, and third pluralities of subscores, wherein the scaled nutrition score is scaled to a calorie reference value; and
generating a nutrition quality ratio by dividing the scaled nutrition score by a number of calories per serving of the food item.

18. The non-transitory computer-readable medium of claim 18, wherein the method further comprises transmitting the nutrition quality ratio to a user device.

19. The non-transitory computer-readable medium of claim 19, wherein the method further comprises:
generating a recommendation regarding the food item based on the nutrition quality ratio; and
transmitting the recommendation to the user device.

20. The non-transitory computer-readable medium of claim 18, wherein the instructions cause the one or more processors to perform the method multiple times for a plurality of different food items.
